(19) United States
(12) Patent Application Publication
(10) Pub. No.: US 2012/0259368 A1
(43) Pub. Date: Oct. 11, 2012

(54) SPACER APPARATUS FOR MANINTAINING INTERSPINOUS SPACING

(75) Inventors: Chang-Hwa YOU, Gunpo-si (KR); Boo-Gyu PARK, Incheon (KR); Yun-Sung KANG, Gunpo-si (KR)

(73) Assignee: Chang-Hwa YOU, Gunpo-si (KR)

(21) Appl. No.: 13/095,215

(22) Filed: Apr. 27, 2011

(30) Foreign Application Priority Data
Apr. 6, 2011 (KR) 10-2011-0031456

(51) Int. Cl. A61B 17/70

(52) U.S. Cl. 606/249

(57) ABSTRACT

A spacer apparatus for maintaining an interspinous spacing is provided. The spacer apparatus includes a first member and a second member. The first member includes a first body section supporting the upper spinous process, a first upper wing section supporting one side of the upper spinous process, a first lower wing section supporting one side of the lower spinous process, and a protrusion laterally extending from the first body section. The second member includes a second body section having an insertion hole through which the protrusion is inserted, a second upper wing section supporting the other side of the upper spinous process, and a second lower wing section supporting the other side of the lower spinous process.

### PRIORITY

This application claims the benefit under 35 U.S.C. §119(a) of a Korean patent application filed on Apr. 6, 2011 in the Korean Intellectual Property Office and assigned Serial No. 10-2011-0031456, the entire disclosure of which is hereby incorporated by reference.

### BACKGROUND OF THE INVENTION

1. Field of the Invention

The present invention relates to a spacer apparatus for maintaining an interspinous spacing. More particularly, the present invention relates to a spacer apparatus for expanding a constricted interspinous spacing of a patient suffering from spinal stenosis and maintaining it constant.

2. Description of the Related Art

The vertebral column is a column that serves to offer stability and balance to a human body. The vertebral column plays an important role to protect a spinal nerve extending from a brain to the limbs, i.e. a spinal cord. The spinal cord passes through a spinal canal, and if stenosis occurs in the spinal canal, spinal stenosis accompanied with nerve compression, an inflammation, and a pain may be generated.

A patent suffering from spinal stenosis should release the compressed spinal cord through surgical treatment. Such a surgical treatment includes a variety of methods such as a vertebral pedicel screw fixation, a prosthesis insertion, in which a spacer prosthesis is inserted between spinous processes in order to maintain an interspinous spacing, and the like. The present invention relates to such a spacer prosthesis for maintaining the interspinous spacing in order to perform an operation on a patient for spinal stenosis.

With the advance of a medical technique, diverse kinds of operations for spinal stenosis and therefore such a spacer prosthesis for maintaining an interspinous spacing are being developed.

Korean Utility Model Registration No. 20-0382167 (hereinafter referred to as a ‘related technology 1’) discloses a conventional spacer prosthesis which includes wing sections fitted around spinous processes and a U-type body. Further, Korean Unexamined Patent Publication Nos. 10-2005-0000425 (‘related technology 2’) and 10-2005-0119791 (‘related technology 3’) disclose spacer prostheses which are adjustable according to a width of a spinous process that varies with every person.

However, since these spacer prostheses of the related art are designed to be fitted and inserted into the vertebra from the posterior side towards the anterior side, upon insertion of the spacer prosthesis, a ligament passing through the posterior of the vertebra may be torn to cause a fatal danger such as fracture of a bone.

Further, while the spacer prostheses of the related technologies 2 and 3 sufficiently hold the most posterior portion of the spinous process, the body sections thereof, fitted between the spinous processes, cannot sufficiently support the middle portion of the spinous process, so that a problem is caused in that the spinous process that has already become atrophied and weakened is continuously subjected to load with the lapse of time. Furthermore, in case of the spacer prosthesis of the related technology 1, while such a problem does not occur in the spacer prosthesis due to having the U-type body section to be fitted between the spinous processes, such a shape may problematically compress the nerve passing along the vertebra.

Moreover, if stenosis also occurs on spinous processes that are adjacent to the pair of spinous processes to which the spacer prosthesis was fitted, another spacer prosthesis is needed to be fitted to those spinous processes. However, according to the related art, it is difficult to further fit a spacer prosthesis in proximity to the already-fitted spacer prosthesis due to its geometrical characteristic. In this case, a problem arises that existing spacer prosthesis should be removed and other means (e.g. vertebral pedicel screw) has to be fitted.

### SUMMARY OF THE INVENTION

Aspects of the present invention are to address at least the above-mentioned problems and/or disadvantages and to provide at least the advantages described below. Accordingly, an aspect of the present invention is to provide a spacer apparatus for maintaining an interspinous spacing that does not cause danger of damaging a ligament or a bone upon operation, is firm and able to be fitted to the vertebrae anatomically suitably.

According to an aspect of the present invention, a spacer apparatus for maintaining an interspinous spacing is provided. The apparatus includes a first member and a second member, the first member having: a first body section located between an upper-side spinous process and a lower-side spinous process so as to support the upper-side spinous process, a first upper wing section extending upwards from the first body section so as to support one side of the upper-side spinous process; a first lower wing section extending downwards from the first body section so as to support one side of the lower-side spinous process, and a protrusion laterally extending from the first body section, the second member having: a second body section having an insertion hole through which the protrusion is inserted, a second upper wing section extending upwards from the second body section so as to support the other side of the upper-side spinous process, and a second lower wing section extending downwards from the second body section so as to support the other side of the lower-side spinous process.

The protrusion of the first member may be provided at an end with an engaging tip that is larger than the other portion of the protrusion, and the insertion hole of the second member may be provided with an engaging step in which the engaging tip is caught.

The second member may be provided with a second fastening hole extending from the back side of the second body section to the insertion hole, and the protrusion of the first member may be provided with a first fastening hole that is formed to correspond to the second fastening hole, and a fastening screw may be further provided to be inserted into the second fastening hole and the first fastening hole from the back side of the second member.

The first upper wing section may be shorter than the first lower wing section, and the second upper wing section may be shorter than the second lower wing section.

A first distance between a first point of the first upper wing section and a second point of the second upper wing section oppositely facing the first point may be longer than a second distance between a third point of the first lower wing section that is spaced apart from the first body section by the same distance as the distance between the first point of the first upper wing section and the first body section, and a fourth point of the second lower wing section that oppositely faces the third point of the first lower wing section.

The second distance between the third point of the first lower wing section and the fourth point of the second lower wing section oppositely facing the third point may become longer as it goes downwards.

According to the present invention, following effects are offered.

First, since a side approach operation is possible, there is no danger of tearing a ligament passing along the posterior of the vertebrae and thus causing fracture of the vertebrae.

Second, since the spacer apparatus is inserted through a lateral side of the vertebrae and is coupled thereto in an interference-fitting manner, despite a divided structure, stable coupling and furthermore coupling stability due to dual coupling are ensured.

Third, since the body section to be fitted between the spinous processes sufficiently supports the middle portion of the spinous process, the spinous process receives less load, so that an interspinous spacing can be stably maintained even with the lapse of time.

Fourth, there is no fear of compressing the nerve due to its anatomically suitable design of the body section.

Fifth, since the lengths of the upper wing section and the lower wing section are asymmetric, so that even when the spacer apparatuses are fitted in series to adjacent spinous processes, interference of the spacer apparatuses with the adjacent spinous processes does not occur.

Sixth, since the geometrical shape of the spacer apparatus is formed to match with the shape of the spinous process, and particularly the upper side thereof is designed to be larger than the lower side thereof, the spacer apparatus can maintain the interspinous spacing with ease while the spinous process being not subjected to much load.

Seventh, sine the side portion has an elastic structure such as a central concave recess the spacer apparatus is resistant to load and has no fear of being broken.

Other aspects, advantages, and salient features of the invention will become apparent to those skilled in the art from the following detailed description, which, taken in conjunction with the annexed drawings, discloses exemplary embodiments of the invention.

### BRIEF DESCRIPTION OF THE DRAWINGS

The above and other aspects, features, and advantages of certain exemplary embodiments the present invention will be more apparent from the following description taken in conjunction with the accompanying drawings, in which:

FIG. 1 is a photograph showing a model of spinous processes according to an exemplary embodiment of the present invention;

FIG. 2 is a perspective view showing a spacer apparatus for maintaining an interspinous spacing according to an exemplary embodiment of the present invention;

FIG. 3 is a perspective view showing a first member of the spacer apparatus according to an exemplary embodiment of the present invention;

FIG. 4 is a perspective view showing a second member of the spacer apparatus according to an exemplary embodiment of the present invention;

FIG. 5 is an exploded perspective view showing the spacer apparatus according to an exemplary embodiment of the present invention;

FIG. 6 is a cross-sectional view taken along line VI-VI of the spacer apparatus of FIG. 2 according to an exemplary embodiment of the present invention;

FIG. 7 is a cross-sectional view taken along line VII-VII of the spacer apparatus of FIG. 2 according to an exemplary embodiment of the present invention; and

FIG. 8 is a view showing the state of plural spacer apparatuses being adapted to the adjacent spinous processes according to an exemplary embodiment of the present invention.

Throughout the drawings, it should be noted that like reference numbers are used to depict the same or similar elements, features, and structures.

### DETAILED DESCRIPTION OF EXEMPLARY EMBODIMENTS

The following description with reference to the accompanying drawings is provided to assist in a comprehensive understanding of exemplary embodiment of the invention as defined by the claims and their equivalents. It includes various specific details to assist in that understanding but these are to be regarded as merely exemplary. Accordingly, those of ordinary skill in the art will recognize that various changes and modifications of the embodiments described herein can be made without departing from the scope and spirit of the invention. In addition, descriptions of well-known functions and constructions may be omitted for clarity and conciseness.

The terms and words used in the following description and claims are not limited to the bibliographical meanings, but, are merely used by the inventor to enable a clear and consistent understanding of the invention. Accordingly, it should be apparent to those skilled in the art that the following description of exemplary embodiments of the present invention is provided for illustration purpose only and not for the purpose of limiting the invention as defined by the appended claims and their equivalents.

It is to be understood that the singular forms “a,” “an,” and “the” include plural referents unless the context clearly dictates otherwise. Thus, for example, reference to “a component surface” includes reference to one or more of such surfaces.

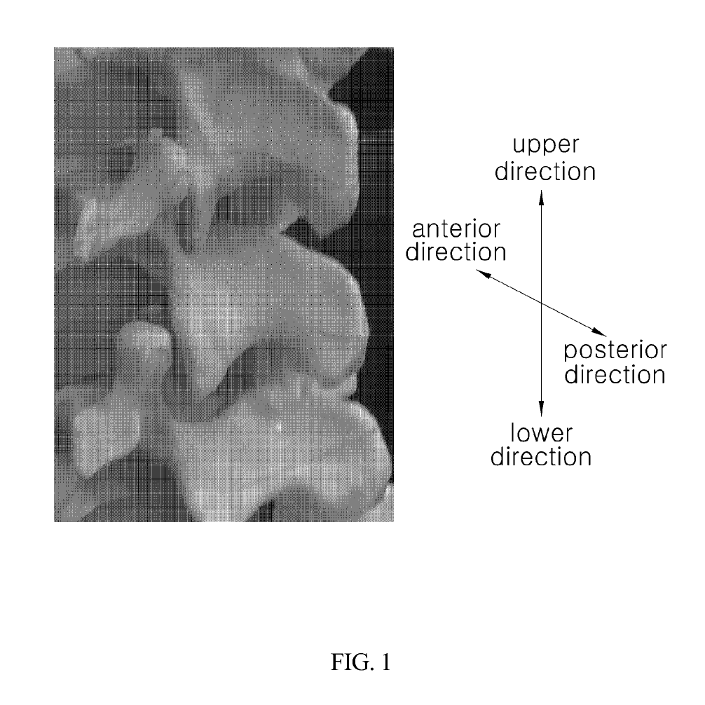

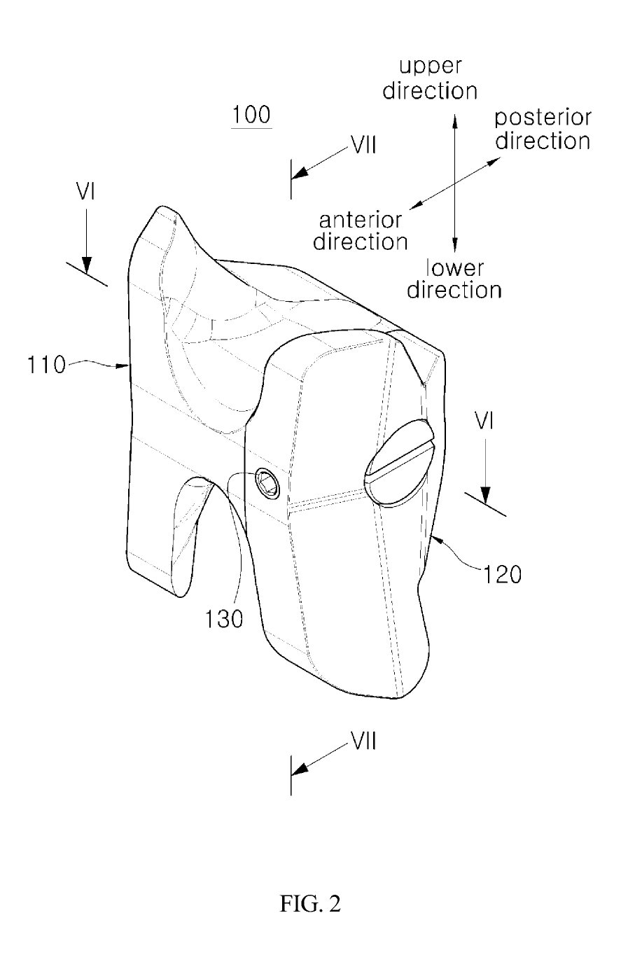

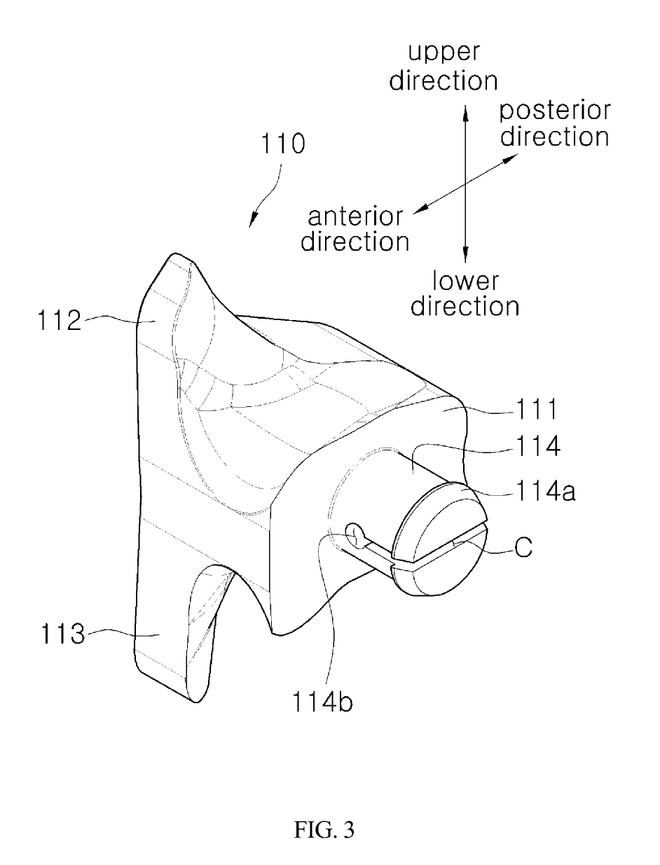

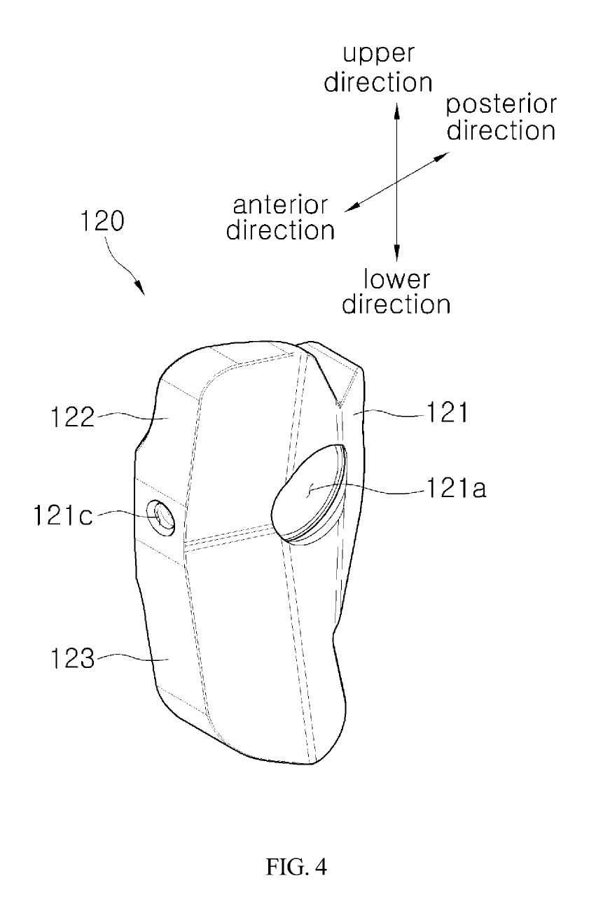

FIG. 1 is a photograph showing a model of spinous processes according to an exemplary embodiment of the present invention. FIG. 2 is a perspective view showing a spacer apparatus for maintaining an interspinous spacing according to an exemplary embodiment of the present invention. FIG. 3 is a perspective view showing a first member of the spacer apparatus according to an exemplary embodiment of the present invention. And FIG. 4 is a perspective view showing a second member of the spacer apparatus according to an exemplary embodiment of the present invention.

Referring to FIGS. 2 to 4, the spacer apparatus 100 for maintaining an interspinous spacing includes a first member 110 and a second member 120, which are coupled by means of a fastening screw 130.

The spacer apparatus 100 is a spacer prosthesis which is inserted into a constricted interspinous spacing between spinous processes of a patient suffering from spinal stenosis so as to maintain the interspinous spacing at a constant interval. As such, the spacer apparatus consists of two members, i.e. the first and second members 110 and 120, so that it is possible to insert the spacer apparatus between spinous processes through side approach without a need of approaching through the posterior of the vertebrae, enabling an operation to be performed without causing a ligament to be damaged.

The first member 110 includes a first body section 111, a first upper wing section 112, a first lower wing section 113, and a protrusion 114.

The first body section 111 is located between adjacent upper and lower spinous processes so as to support the upper spinous process, thereby maintaining inerspinous spacing between the upper and lower spinous processes.

As shown in FIG. 1, the spinous process has the shape in which the lower portion thereof is thicker than the upper portion thereof, and the posterior side thereof is thicker than the anterior side thereof. Here, the direction as mentioned is indicated with reference to a human body, so that the upper and lower direction means the lengthwise direction of the vertebrae, the anterior direction means the direction facing abdomen, and the posterior direction means the direction facing the back.

The first body section 111 of the first member 110 has the shape that is capable of pressing the lower spinous process while supporting the upper spinous process. That is, the first body section 111 has the shape of an integrated piece which is provided so that the upper and lower surfaces thereof are round such that the posterior surface thereof is recessed deeper and wider than the anterior surface thereof, which allows the spinous processes to be supported as it stands without stimulating the upper and lower spinous processes.

The first upper wing section 112 extends upwards by a certain distance from one side of the first body section 111, and the first lower wing section 113 extends downwards by a certain distance from one side of the first body section 111.

The upper and lower wing sections 112 and 113 is integrated with the first body section 111, seating between adjacent spinous processes, as to extend upwards and downwards by certain distances, respectively, thereby supporting the upper and lower spinous processes while preventing the respective spinous processes from moving laterally. A detailed feature of the first upper and lower wing sections 112 and 113 will be described later.

Next, the protrusion 114 is a feature that extends laterally from the first body section 111 in order to fasten the first member 110 to the second member 120 to be described later.

That is, the protrusion 114 has the shape of a cylinder that protrudes from the side of the first body section 111 so as to be inserted into an insertion hole 121a of the second member 120 that is formed to correspond to the protrusion.

The protrusion 114 is provided at an end with an engaging tip 114a that is inclined and is larger than the other portion of the protrusion 114. The engaging tip 114a is formed into a divided form having two legs with a slit C formed therebetween. Such construction is a feature to allow the engaging tip 114a, a diameter of which is larger than the insertion hole 121a to be described later, to be inserted into the insertion hole 121a, so that upon insertion, the protrusion 114 is inserted into the insertion hole 121a while the distance between the two legs of the engaging tip is made narrow in the slit, and after insertion, the two legs are outstretched so that the engaging tip 114a is fixedly caught in an engaging step 121b of the insertion hole 121a to be described later.

The slip C of the protrusion 114 is provided with a first fastening hole 114b which is formed to correspond to a second fastening hole 121c of the second member 120 to be described later. The fastening screw 130 passes through the second fastening hole 121c and into the first fastening hole 114b from the back side of the second member 120.

Next, the second member 120 includes a second body section 121, a second upper wing section 122, and a second lower wing section 123.

The second body section 121 is located between adjacent upper and lower spinous processes so as to press the lower spinous process while supporting the upper spinous process. The second body section is formed to correspond to the first body section 111, so that upon coupling, the second body section and the first body section form an integrated shape. That is, the second body section 121 has the shape of an integrated piece with the first body section 111 so that the upper and lower surfaces thereof are round such that the posterior surface thereof is recessed deeper and wider than the anterior surface thereof, which allows the spinous processes to be supported as it stands without stimulating the upper and lower spinous processes.

The second body section 121 is provided with the insertion hole 121a through which the protrusion 114 of the first member 110 is inserted so as to be coupled to the second body section.

The insertion hole 121a is formed to correspond to the protrusion 114 of the first member 110, and is provided at an end with the engaging step 121b, in which the engaging tip 114a of the protrusion 114 is inserted into the insertion hole 121a in a narrowed state and then is outstretched and fixedly caught.

The second body section 121 is provided with a second fastening hole 121c to correspond to the first fastening hole 114b, thereby allowing the fastening screw 130 to be inserted into the first fastening hole 114b through the second fastening hole 121c from the back side of the second body section 120.

The second upper wing section 122 extends upwards by a certain distance from one side of the second body section 121, and the second lower wing section 123 extends downwards by a certain distance from one side of the second body section 121, thereby supporting the upper and lower spinous processes while preventing the respective spinous processes from moving laterally.

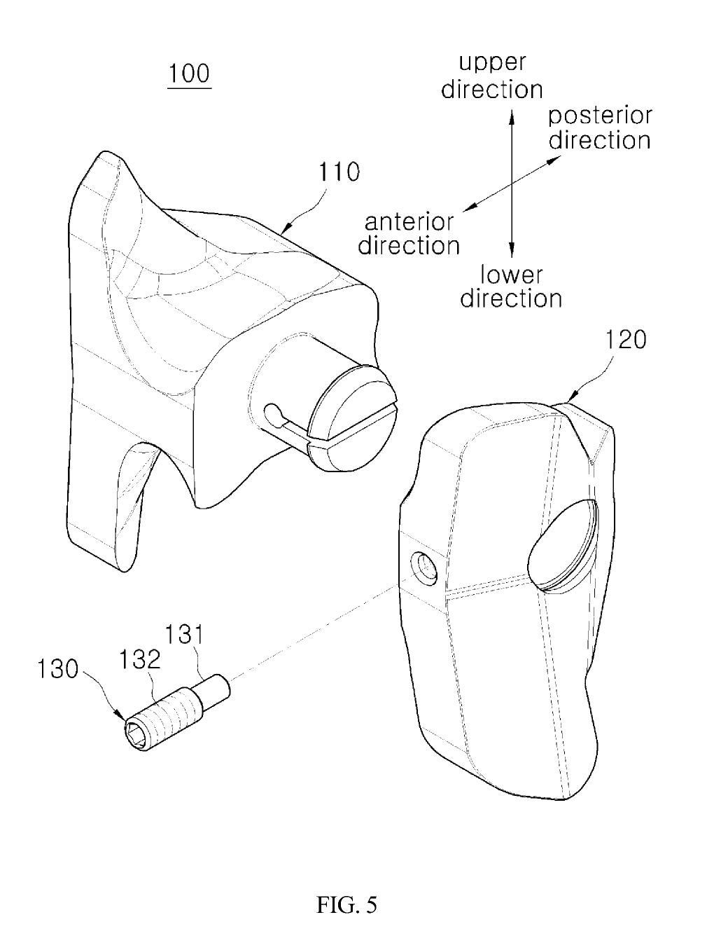

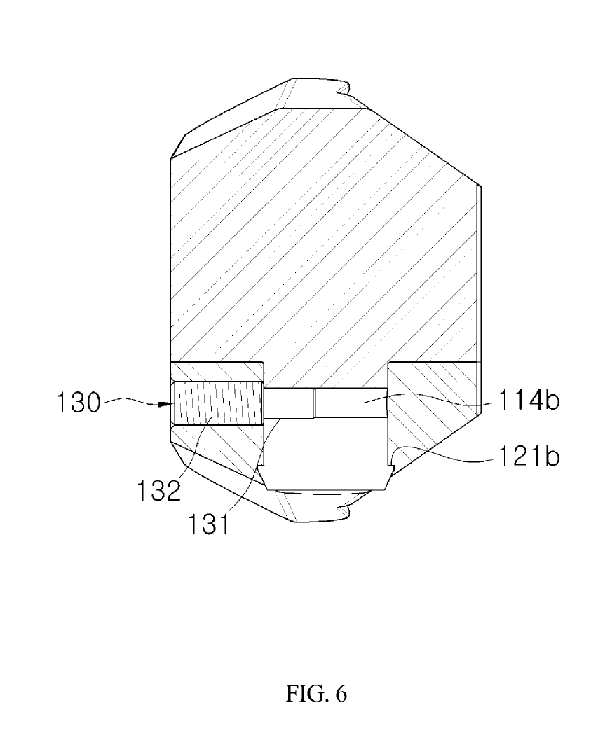

FIG. 5 is an exploded perspective view showing the spacer apparatus according to an exemplary embodiment of the present invention, and FIG. 6 is a cross-sectional view taken along line VI-VI of the spacer apparatus of FIG. 2 according to an exemplary embodiment of the present invention.

In the spacer apparatus 100 of the present invention, the first and second members 110 and 120 are coupled together through insertion coupling, and then the fastening screw 130 is screwed thereto for further firm coupling.

In assembly, the protrusion 114 extending from the first body section 111 of the first member 110 is inserted into and coupled to the insertion hole 121a of the second body section 121 of the second ember 120. That is, separate first and second members 110 and 120 can approach the interspinous spacing from the side of the spinous processes without interfering with a ligament near the spinous processes, and be coupled together through insertion coupling.

Since the engaging tip 114a of the protrusion 114 of the first member has a diameter larger than the other portion of the protrusion 114, and the diameter of the second member 120 is formed to correspond to the diameter of the other portion of the protrusion 114, the protrusion cannot be inserted into the second member as it is. However, the slit C makes it possible for the protrusion to be inserted into the second member. That is, when inserted, the protrusion 114 is inserted into the insertion hole 121a with its two legs gathering in the slit, and after inserted, the two legs becomes outstretched in the slit so that the protrusion 114 is caught in the engaging step 121b of the insertion hole 121a.

The engaging tip 114a of the protrusion 114 is widen and fixedly caught in the engaging step 121b of the insertion hole 121a, thereby completing coupling between the first and second members.

Additionally, the fastening screw 130 is provided for secondary coupling. The fastening screw 130 is configured to have a first fastening portion 131 and a second fastening portion 132, wherein a diameter of the first fastening portion 131 is made smaller than the diameter of the second fastening portion 132. Thus, in conformity with this construction, the diameter of the first fastening hole 114b of the first member 110 may preferably be made smaller than the diameter of a second fastening hole 121c of the second member 120. Further, the tip portions of the first and second fastening portions 131 and 132 may preferably be made inclined in order to carry out smooth coupling.

The second fastening portion 132 may preferably have a threaded part so as to be screwed into the second fastening hole 121c. In this case, when the fastening screw 130 is fastened to the first and second members 110 and 120 coupled together, the first fastening portion 131 and the second fastening portion 132 are smoothly screw-coupled into the first fastening hole 114b and the second fastening hole 121c, respectively.

Here, the first fastening portion 131 has the diameter to correspond to the diameter of the first fastening hole 114b, so that when the protrusion 114 is inserted into the insertion hole 121a, the diameter of the first fastening hole 114b is further narrowed due to constriction of the slit C, so that upon insertion, the first fastening portion 131 widens the first fastening hole 114b, thereby performing interference-coupling of the first fastening portion 131, which allows the protrusion 114 to be outstretched and come into close contact with the insertion hole 121a.

With further firm coupling through fastening the fastening screw 130 to the first fastening hole while the first and second members 110 and 120 are in a coupled state, the first and second member 110 and 120 are prevented from being disconnected.

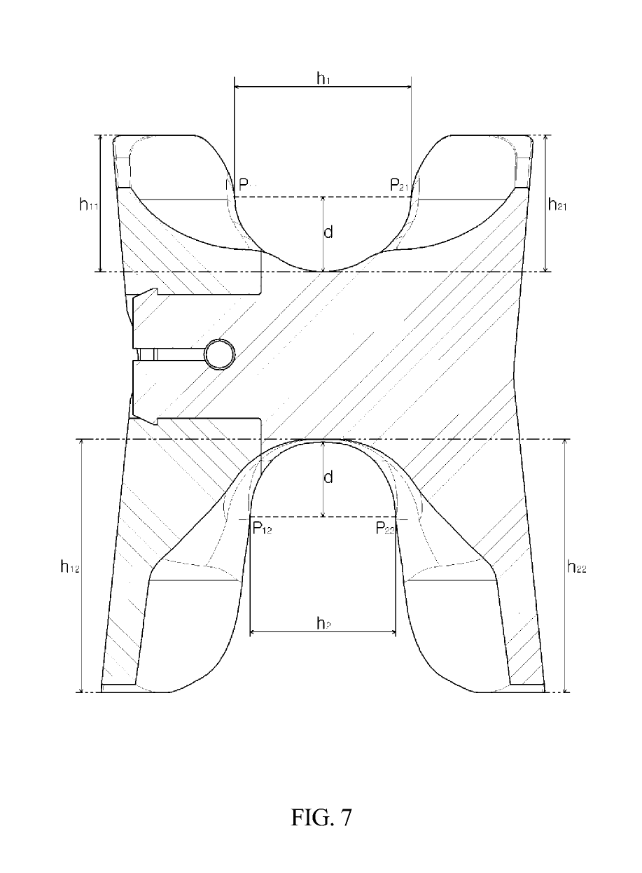

FIG. 7 is a cross-sectional view taken along line VII-VII of the spacer apparatus of FIG. 2 according to an exemplary embodiment of the present invention. A feature of the spacer apparatus will be further described with reference to FIG. 7.

In the spacer apparatus 100 of the present invention in which the first and second members 110 and 120 are coupled to each other and further using the fastening screw 130, the first upper wing section 112 is shorter than the first lower wing section 113, and the second upper wing section 122 is shorter than the second lower wing section 123.

That is, as shown in FIG. 7, the length h11 of the first upper wing section 112 is longer than the length h12 of the first lower wing section 113, and the length h21 of the second upper wing section 122 is longer than the length h22 of the second lower wing section 123.

Such a construction is caused by the shape of the spinous process and is also provided for the purpose of additionally fitting another spacer apparatus to the spinous processes neighboring the spinous processes to which the spacer apparatus was already fitted.

As shown in FIG. 1, the spinous process is schematically shaped so that the upper portion thereof is narrower and sharper than the lower portion thereof. Thus, if such configuration is not taken into consideration, so that the lengths of the upper wing sections 112 and 122 and the lower wing sections 113 and 123 are the same, spacer apparatuses cannot be fitted in series to the adjacent spinous processes.

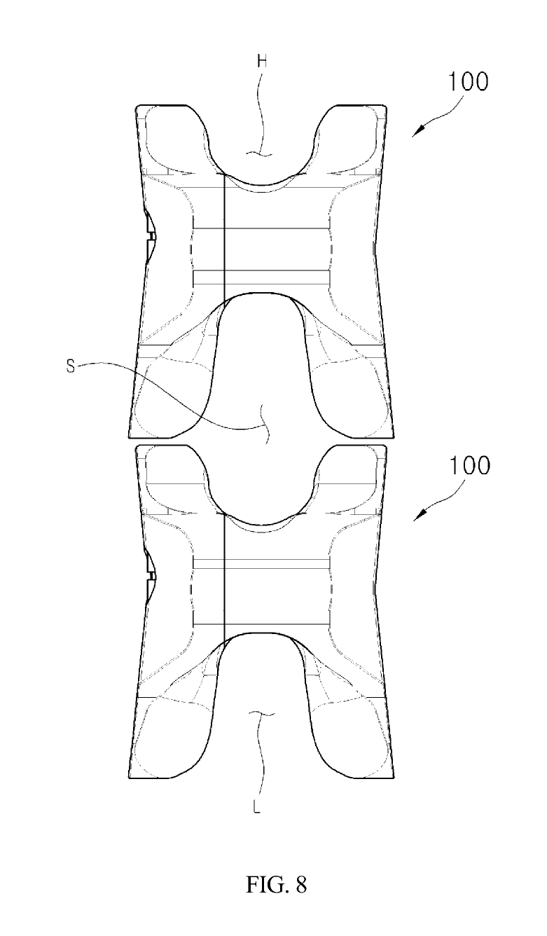

However, the spacer apparatus 100 of the present invention is configured such that plural spacer apparatuses can be safely fitted in series to the adjacent spinous processes without interference, so that the length and shape of the upper wing sections 112 and 122 and the lower wing sections 113 and 123 are formed to correspond to the shape of the spinous process. That is, as can be seen from FIG. 8, the spacer apparatus 100 of the present invention has the construction in which when two spacer apparatuses are fitted in series to the adjacent spinous processes, a space is defined between the two apparatuses in order to safely support the spinous process therein.

Further, in the spacer apparatus 100 of the present invention, a first distance between a first point of the first upper wing section and a second point of the second upper wing section oppositely facing the first point is configured to be longer than a second distance between a third point of the first lower wing section that is spaced apart from the first body section by the same distance as the distance between the first point of the first upper wing section and the first body section, and a fourth point of the second lower wing section that oppositely faces the third point of the first lower wing section.

That is, as shown in FIG. 7, the first distance h1 between the first point P11 on the back of the first upper wing section 112 and the second point P21 on the back of the second upper wing section 122 oppositely facing the first point P11 is configured to be longer than the second distance h2 between the third point P12 on the back of the first lower wing section 113 that is spaced apart from the first body section 111 by the same distance as the distance d between the first point P11 on the back of the first upper wing section 112 and the first body section 111, and the fourth point P22 on the back of the second lower wing section 123 that oppositely faces the third point P12 on the back of the first lower wing section 113.

Such a configuration is caused by the shape of the spinous process.

In the spacer apparatus 100 of the present invention, the upper wing sections 112 and 122 support the upper spinous process and the lower wing sections 113 and 123 support the lower spinous process. As shown in FIG. 1, the schematic shape of the spinous process is shaped so that the upper portion thereof is narrower and shaper than the lower portion thereof.

Thus, if such a configuration is not taken into consideration, so that the width between the upper wing sections 112 and 122 and the width between the lower wing sections 113 and 123 are the same, the spacer apparatus cannot properly support the upper and lower spinous processes.

That is, as shown in FIG. 8, the width between the upper wing sections 112 and 122 is configured to be larger than the width between the lower wing sections 113 and 123, so that an upper space H defined by the upper wing sections 112 and 122 is made slightly thinner and easier, and the a lower space L defined by the lower wing sections 113 and 123 is made slightly deeper and shaper, thereby allowing an upper spinous process to be supported in the upper space H to correspond to the shape of a lower portion of the upper spinous process and allowing a lower spinous process to be supported in the lower space L to correspond to the shape of an upper portion of the lower spinous process.

Thereby, with the above-mentioned construction, the spacer apparatus of the present invention can safely support the spinous processes.

Next, the second distance between the third point of the first lower wing section and the fourth point of the second lower wing section oppositely facing the third point may become longer as it goes downwards.

That is, as shown in the drawing, the distance between the lower wing sections 113 and 123 has an inclined form which is longer as it goes downwards. This is also caused by the shape of the spinous process, and also because the upper portion of the spinous process is made narrower and sharper than the lower portion thereof as shown in FIG. 1.

Thereby, with the above-mentioned construction, the spacer apparatus of the present invention can safely support the spinous processes.

Further, as shown in FIG. 7, the portions defined by the upper wing sections 122 and 122 and the lower wing sections 113 and 123 are shaped like a bow in which the body sections 111 and 121 are slightly recessed, so that the spacer apparatus of the present invention becomes strongly resistant to load.

The spacer apparatus 100 of the present invention is an apparatus for maintaining an interspinous spacing between spinous processes of a patient suffering from spinal stenosis. The interspinous spacing between the spinous processes of the patient is essentially apt to continuously narrow due to atrophy of the vertebrae. Thus, the spacer apparatus fitted between the spinous processes is increasingly subjected to load with the lapse of time. The bow like shape of the spacer apparatus makes it possible to resist such load.

As set forth before, the spacer apparatus of the present invention has the anatomically suitable structure so that it is adapted to the human body in a safe, proper manner.

Further, the spacer apparatus of the present invention may preferably be formed of an anatomically suitable material such as titanium, plastics, metal, or a bio material such as bio polyetheretherketone (Peek), or the like.

While the invention has been shown and described with reference to certain exemplary embodiments thereof, it will be understood by those skilled in the art that various changes in form and details may be made therein without departing from the scope and spirit of the invention as defined by the appended claims and their equivalents.

What is claimed is:

1. A spacer apparatus for maintaining an interspinous spacing, the spacer apparatus comprising:
a first member and a second member,
the first member having: a first body section located between an upper-side spinous process and a lower-side spinous process so as to support the upper-side spinous process; a first upper wing section extending upwards from the first body section so as to support one side of the upper-side spinous process; a first lower wing section extending downwards from the first body section so as to support one side of the lower-side spinous process; and a protrusion laterally extending from the first body section,
the second member having: a second body section having an insertion hole through which the protrusion is inserted; a second upper wing section extending upwards from the second body section so as to support the other side of the upper-side spinous process; and a second lower wing section extending downwards from the second body section so as to support the other side of the lower-side spinous process.

2. The spacer apparatus of claim 1, wherein the protrusion of the first member is provided at an end with an engaging tip that is larger than the other portion of the protrusion, and the insertion hole of the second member is provided with an engaging step in which the engaging tip is caught.

3. The spacer apparatus of claim 2, wherein the second member is provided with a second fastening hole extending from the back side of the second body section to the insertion hole, wherein the protrusion of the first member is provided with a first fastening hole that is formed to correspond to the second fastening hole, and wherein the spacer apparatus further comprise a fastening screw provided to be inserted into the second fastening hole and the first fastening hole from the back side of the second member.

4. The spacer apparatus of claim 1, wherein the first upper wing section is shorter than the first lower wing section, and the second upper wing section is shorter than the second lower wing section.

5. The spacer apparatus of claim 1, wherein a first distance between a first point of the first upper wing section and a second point of the second upper wing section oppositely facing the first point is longer than a second distance between a third point of the first lower wing section that is spaced apart from the first body section by the same distance as the distance between the first point of the first upper wing section and the first body section, and a fourth point of the second lower wing section that oppositely faces the third point of the first lower wing section.

6. The spacer apparatus of claim 1, wherein the second distance between the third point of the first lower wing section and the fourth point of the second lower wing section oppositely facing the third point becomes longer as it goes downwards.
